(19) United States
(12) Patent Application Publication
(10) Pub. No.: US 2006/0280768 A1
(43) Pub. Date: Dec. 14, 2006

(54) Meniscal repair device and method

(76) Inventors: Julia Hwang, Wayland, MA (US); Stephanie Kladakis, Watertown, MA (US); Sudhakar Kadiyala, Newton, MA (US); Steven Bowman, Sherbom, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/151,615

(22) Filed: Jun. 13, 2005

(51) Int. Cl. A61F 2/00

(52) U.S. Cl. 424/423

(57) ABSTRACT

Methods and apparatus for treating meniscal tissue damage are disclosed, including a biocompatible meniscal repair device comprising a stent. The tissue repair device is adapted to be placed in contact with a defect in the meniscus and can preferably provide a structure for supporting meniscal tissue and/or encouraging tissue growth through contact with vascularized portions of the meniscus or as a conduit for introduction of exogenous healing therapies.

### BACKGROUND OF THE INVENTION

The present invention generally relates to methods and apparatus for repairing meniscal defects, and in particular to tissue repair devices having an enhanced ability to encourage meniscal repair.

The meniscus is specialized tissue found between the bones of a joint. For example, in the knee the meniscus is a C-shaped piece of fibrocartilage which is located at the peripheral aspect of the joint between the tibia and femur. This tissue performs important functions in joint health including adding joint stability, providing shock absorption, and delivering lubrication and nutrition to the joint. As a result, meniscal injuries can result in debilitating conditions including degenerative arthritis.

Meniscus injuries, and in particular tears, are a relatively common injury. Such injuries can result from a sudden twisting-type injury such as a fall, overexertion during a work-related activity, during the course of an athletic event, or in any one of many other situations and/or activities. In addition, tears can develop gradually with age. In either case, the tears can occur in either the outer thick part of the meniscus or through the inner thin part. While some tears may involve only a small portion of the meniscus, others affect nearly the entire meniscus.

Unfortunately, a damaged meniscus is unable to undergo the normal healing process that occurs in other parts of the body. The peripheral rim of the meniscus at the menisco-synovial junction is highly vascular (red-red zone) whereas the inner two-thirds portion of the meniscus has limited vascularity with the inner one-third being completely avascular (white-white zone). The small transition region between the red-red zone and the white-white zone is called the red-white zone. Degenerative or traumatic tears to the meniscus which result in partial or complete loss of function frequently occur in the white-white zone where the tissue has little potential for regeneration. Such tears result in severe joint pain and locking, and in the long term, a loss of meniscal function leading to osteoarthritis.

Although several treatments currently exist for meniscal injuries, the treatment options provide little opportunity for meniscal healing or regeneration. The majority of meniscal injuries are treated by removing the unstable tissue during a partial meniscectomy. Once the tissue is removed no further treatment is conducted. Most patients respond well to this treatment in the short term but often develop degenerative joint disease several years (i.e., after more than about 10 years) post operatively. The amount of tissue removed has been linked to the extent and speed of degeneration. When the majority of the meniscal tissue is involved in the injury, a total meniscectomy is conducted. If the patient experiences pain after a total meniscectomy without significant joint degeneration, a secondary treatment of meniscal allografts is possible. The use of allografts is limited by tissue availability and by narrow indications.

For meniscal tears that are located in vascularized areas of the meniscus, the tears are often repaired with suture or equivalent meniscal repair devices. While these repairs are successful in approximately 60-90% of the cases, the percentage of injuries which meet the criteria to be repaired is 15% or less. Repair criteria are based not only on vascularity and type of tear but also stability and integrity of the meniscus, stability of the knee and patient factors such as age and activity. If the repair does fail, the next possible course of treatment is either a partial or total meniscectomy.

Trephination is a known method used to promote vascular ingrowth to the site of the meniscal defect. In this technique, small holes are made in the meniscus with a punch (U.S. Pat. No. 6,387,111), a needle or trephine. The holes are to allow a path for vascular ingrowth. The current invention differs from this technique in that this invention describes placing a material into the channel to promote vascular and cellular ingrowth while also having the potential to maintain patency.

Accordingly, there continues to exist a need in this art for novel tissue repair devices capable of encouraging meniscal tissue regeneration, as well as, methods for using such tissue repair devices particularly in the avascular portions of the meniscus.

### BRIEF DESCRIPTION OF THE DRAWINGS

FIG. 1a depicts the menisci of the knee.

FIG. 1b shows a cross-sectional view of the meniscus.

FIGS. 2a, 2b, 2c depict various paths for repair of a meniscal tear.

FIGS. 3a, 3b, 3c, 3d, 3e, 3f, 3g, and 3h depict various designs of suitable meniscal coring or slicing patterns.

FIGS. 4a and 4b show cross-sectional views of a meniscal tear repair using one embodiment of the device of this invention.

FIG. 5 shows an alternate embodiment of this invention wherein a solid or non-solid material is used in the meniscal repair channel.

### SUMMARY OF THE INVENTION

This invention is generally related to methods and apparatus for treating meniscal tissue damage. The tissue repair device and method is adapted to be placed in contact with a defect in the meniscus and can preferably provide a structure for supporting meniscal tissue and/or encouraging tissue growth through contact with vascularized portions of the meniscus or as a conduit for introduction of exogenous healing therapies.

In one embodiment, the invention relates to a biocompatible meniscal repair device, comprising:

a biocompatible tissue repair stent adapted to be placed in contact with a defect in a meniscus and adapted to contact a vascularized portion of the meniscus and communicate biological materials from the vascularized portion of the meniscus to the tissue defect in the meniscus.

In another embodiment, the invention relates to method of surgically repairing meniscal defects, comprising:

providing a tissue repair stent; and
positioning a first end of the tissue repair stent in contact with the defect in the meniscus while positioning a second end of the stent in contact with a vascularized portion of the meniscus, wherein the stent allows blood, cells or nutrients to migrate from the vascularized portion of the meniscus to the defect in the meniscus and thereby encourage healing of the meniscus.

In yet another embodiment, the invention relates to a method of surgically repairing meniscal defects, comprising:

creating a channel or slice in the meniscus between the defect in the meniscus and a vascularized portion of the meniscus; and

providing a material in the channel or slice to permit controlled patency of the channel or slice to enhance migration of blood, cells and nutrients from the vascularized portion of the meniscus to the defect in the meniscus and thereby encourage healing of the meniscal defect.

### DETAILED DESCRIPTION OF PREFERRED EMBODIMENTS OF THE INVENTION

An objective of this invention is to enhance the repair of meniscal tears or defects.

The meniscus is specialized tissue found between the bones of a joint. For example in the knee, the meniscus is a C-shaped piece of fibrocartilage which is located at the peripheral aspect of the joint between the tibia and femur. Each knee has two menisci, one medial and one lateral. Together they cushion the joint by distributing downward forces outward and away form the central anchor points of the menisci.

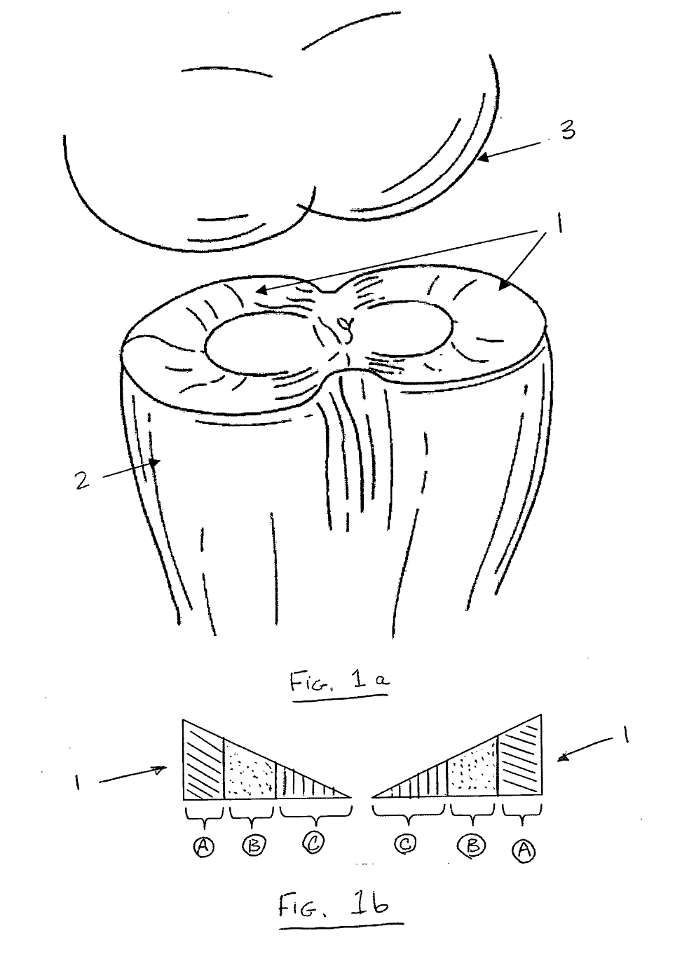

FIG. 1a is a representation of typical menisci 1 situated between the tibia 2 and the femur 3 (femoral chondyle shown). FIG. 1b is a presentation of idealized cross-sections of a meniscus 1 showing the three distinct regions of the menisci, the red-red zone \textcircled{A} (highly vascularized); the red-white zone \textcircled{B} (partially vascularized); and the white-white zone \textcircled{C} (avascular). It is in the red-white and white-white regions of the meniscus that the devices and the methods of this invention have their greatest benefit.

FIGS. 2a, 2b, and 2c show meniscus 1 with tears 20 and various channels 30,40, and 50 directed to meniscal tears 20.

In one embodiment, the invention comprising creating a channel from the vascular zone or periphery of the meniscus to the meniscal defect site. The purpose is for cells, nutrients, and blood supply from another part of the tissue to have easier access to the defect site to enhance healing of damaged tissue. This invention is applicable for all types of tears in both the medial and lateral meniscus and also can be used along with other meniscal repair devices (or sutures) and/or implants that are designed to regenerate tissue such as biocompatible scaffolds and devices known in the art such as extracellular matrices (including small intestine submucosa (SIS) and collagen based scaffolds), polymeric materials (including PLA, PGA) and elastomeric materials; all such materials may be the same as some of the materials hereinafter described as suitable for use in the device of this invention.

In some embodiments, a device will be placed in the channel to facilitate growth of vasculature to the defect site and/or used to preserve channel patency. In other case, the shapes of the coring itself is sufficient to keep an open channel or alternately, once the channel is cored or sliced, anti coagulating, enzymatic or gel materials are added to maintain the openness of the channel.

The channel could be created by coring out tissue or by cutting or slicing tissue without any removal of tissue. This can be achieved by an inside-out technique (i.e., from the inner portions of the meniscus to or through the outer periphery of the meniscus) or an outside-in technique (i.e., from the outer periphery of the meniscus to the inner portions of the meniscus). The channel may be very small to minimize any damage to the meniscus or it may be large to help maintain the blood flow. Multiple channels or only one channel may be created. It is also possible to have multiple channels radiating from one channel or multiple channels radiating from the defect site. The shape of the channel need not be circular in cross-section. For example, it could also be rectangular or oval.

FIGS. 2a, 2b, and 2c depict the wide variety of paths a channel may take in the meniscus 1 to reach meniscal tear 20. More specifically, FIG. 2a shows, separate independent channels 30, 40, and 50 to tear 20. FIG. 2b shows two channels 30 and 50 joining a third channel 40 to treat meniscal tear 20 . Finally, FIG. 2c shows possible orientations of channels 30, 40, and 50 to meniscal tear 20. Thus, the location of the channel may not only be the shortest route from the defect to the periphery of the meniscus. For example, if the tear is a vertical longitudinal tear, the channel need not be to the periphery in a direction orthogonal to the defect. It is also possible to have the channel directed to the horns of the meniscus 2 where there is more vascular tissue present. Thus, there are a multitude of potential channel paths according to this invention not limited to any specific examples provided herein.

As noted above, the shape of the channel need not be circular in cross-section. For example, it could also be rectangular or oval. When forming a channel, an oval shape might be desirable to minimize stress risers due to the cutting process. It addition, the cutting tool make have the alternate shapes as cross sections: ▭ or + or >. Additionally, it is desirable that the coring or slicing pattern used in creating a channel does not contribute to further propagation or degeneration of the meniscal tear.

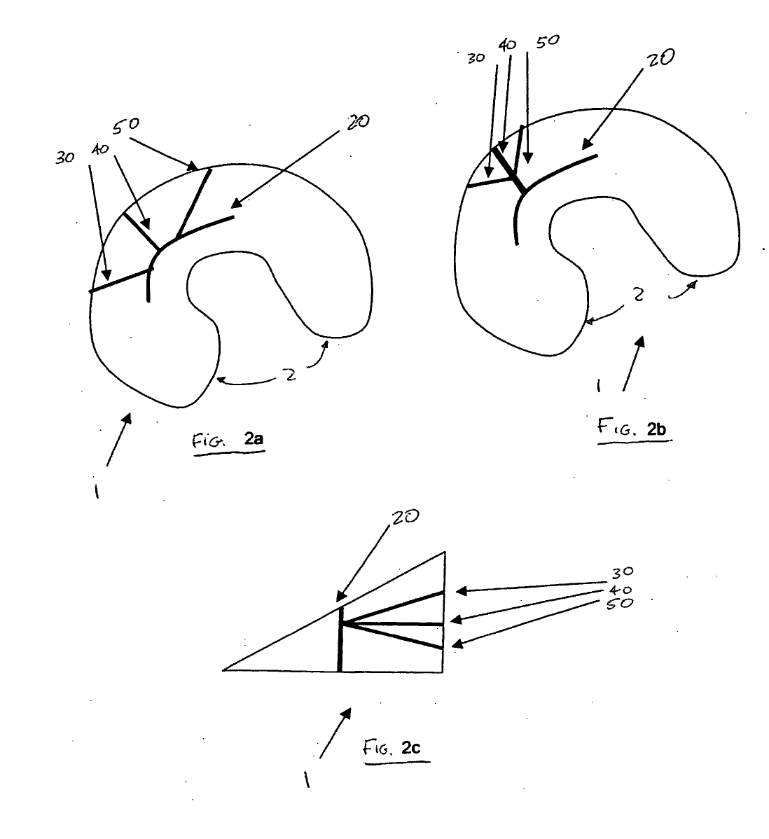

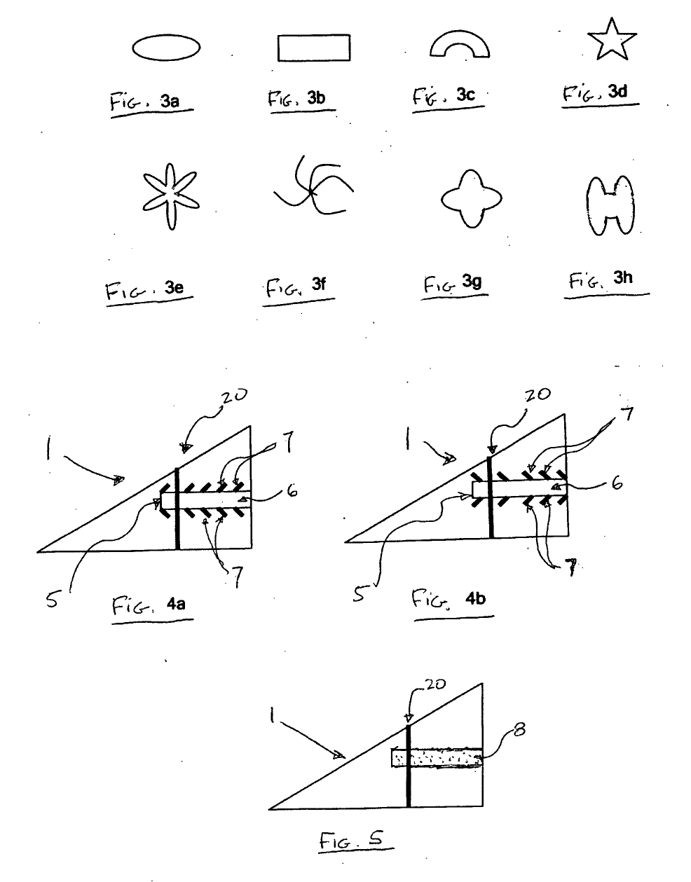

FIGS. 3a to 3h contain additional representative examples of coring or slicing designs. FIG. 3a represents an elliptical shape; FIG. 3b a rectangular shape; FIG. 3c a semi-ring shape; FIG. 3 d a star shape; FIG. 3e an asterisk-like shape; FIG. 3f a free-form, star-like shape; FIG. 3g a cross shape; and FIG. 3h a dumbbell shape.

After or during the formation of a channel whether by coring or slicing, it is desirable to maintain the patency (openness) of the channels to allow flow of cells, blood or nutrients to the defect or tear by adding an anticoagulant, enzyme, or gel. The anticoagulant or enzyme, may be present in a solid, liquid or semi-solid carrier, such as a gel.

Examples of suitable anticoagulants included but are not limited to acid citrate dextrose (ACD), citrate phosphate dextrose (CPD), citrate, heparin, potassium oxalate, potassium citrate, potassium ethylene diamine tetraacetic acid (EDTA) and combinations thereof.

Example of suitable enzymes include, but are not limited to, collagenase, chondroitinase, trypsin, elastase, hyaluronidase, peptidase, thermolysin, matrix metalloproteinase, gelatinase, protease and combinations thereof.

The device of this invention that maintains the blood access to the defect and may also contain a bioactive that enhances tissue repair. The device may be resorbable or non-resorbable or a combination thereof and may be a liquid, gel, solid, or any combination of these three forms. The device can also be made out of multiple types of materials. The device can be a solid material or have a lumen such as a tube or stent. For example, the inner lumen of the device may be composed of one type of material that may help facilitate blood flow and cell migration and the outer surface be made from another type of material that have adhesive and fixation properties. The composition of the tube can also be different along the length of the tube. The tube may be made out of materials with different resorption properties such that a fast resorbing material is surrounded by a slow resorbing material. With time, this device will develop a larger lumen to maintain access to blood supply. The wall of this stent may be a solid and may be porous or nonporous. The stent may also be a mesh or mesh like structure. The device may be stiff or it may be flexible.

In the embodiment of the invention when solid devices are used, the solid device is placed in the channel to facilitate migration of cells and blood supply to the defect. The device may also span the tear and may be used to repair the tear. The device may be placed by the same instrument used to make the channel or it may be placed following the channel placement. The device may also be flexible to allow delivery through cannulas. The device may be a stent or stent-like as known and used in the cardiovascular fields and inserted via the same type of instrumentation that is known in the state of art for cardiovascular applications. This stent may be expandable and may be fixed in place. The stent can also have bioactive coatings that help enhance a vascular response. The device may have the same shape as the channel or a different shape, that may include circular, oval, spiral, star or other shapes such as those represented in FIGS. 3a to 3h. The device may also be sponge, foam or scaffold that does not contain an unobstructed lumen, i.e., blood or nutrients are allowed to flow or migrate across or through the device such as through wicking. The device in such an embodiment, can also be made of the same materials of traditional scaffolds or tissue repair devices that eventually are resorbed or absorbed by the body and which eventually allow regrowth of tissue in or through the device.

The device may be made of a shape memory material such as alloys or polymers. Shape memory alloys are a class of metal alloys that have the ability to remember their original shape. When the metals are mechanically deformed from their original shape (for example via bending and twisting through minimally invasive techniques and cannulas), they regain their initial form. Some examples of shape memory alloys include NiTi (Nitinol), CuZnAl, and CuAlNi.

The shape memory effect also exists for polymers and is a result of polymer's structure and morphology. Shape memory polymers are usually phase separated block copolymers with hard and soft segments that have distinct melting points or glass transition temperatures. In general, the melting point or glass transition temperature of the soft segment is substantially less than the melting point or glass transition temperature of the hard segment. For shape memory polymers, the material returns to original shape by heating above the melting point or glass transition temperature of the soft segment but below the melting point or glass transition temperature of the hard segment. Shape memory behavior has been shown in polyurethanes and in resorbable polymers such as PLA-PCL.

FIGS. 4a and 4b depict device 5 in the form of a stent with lumen 6 and barbs 7 that help to maintain device 5 across the meniscal tear 20. More specifically, FIG. 4a is more suited for an outside-in technique for placement in the meniscus due to the nature of the direction barbs 7 are positioned. FIG. 4b, is more suited for inside-out placement of device 5 due to the nature of the direction the barbs are positioned. While device 5 is shown with barbs 7 and lumen 6, it should be appreciated that device 5 may be solid in form with sufficient porosity (such as with a sponge foam) to permit cells, nutrients, and blood to travel through device 5 to meniscal tear 20 by actions such as by wicking. Also barbs 7 need not be present.

One of the bioactives that can be incorporated with the device of this invention is a growth factor like rhGDF-5 or a combination of growth factors. An anti-fibrotic growth factor may be incorporated in the lumen of the device to prevent occlusion or an angiogenic growth factor may be incorporated in the lumen to promote vasculature. It is also possible to inject bioactives or cells into the lumen of the device. Bioactives for this application include and are not limited to bone marrow, blood, platelet rich plasma, and minced tissue.

The materials suitable for use in the device of this invention can vary, as long as they provide sufficient strength to withstand the stresses required to support a repaired meniscal tear. Moreover, the materials used to construct the device are preferably biocompatible and cause little or no foreign body reaction. The device can be constructed of the same or different biocompatible materials and be any number of tissue repair materials known in the art.

Sufficient strength and physical properties of the device can be developed or achieved through the selection of materials used to form the device, and the process used to manufacture the device. In an exemplary embodiment, the device is formed from a bioresorbable or bioabsorbable material, and more preferably from a bioresorbable or bioabsorbable material that has the ability to resorb in a timely fashion in the body environment. For example, bioresorbable or bioabsorbable material can preferably resorb in less than a year. For the purposes of this invention, the terms “bioresorbable” and bioabsorbable” are intended to be used interchangeably and denote a material that is excreted from a body through typical physiological pathways.

In one embodiment of the present invention, the device can be formed from a biocompatible polymer. A variety of biocompatible polymers can be used according to the present invention. The biocompatible polymers can be synthetic polymers, natural polymers or combinations thereof. As used herein the term “synthetic polymer” refers to polymers that are not found in nature, even if the polymers are made from naturally occurring biomaterials. The term “natural polymer” refers to polymers that are naturally occurring.

In embodiments where the device includes at least one synthetic polymer, suitable biocompatible synthetic polymers can include polymers selected from the group consisting of aliphatic polyesters, poly(amino acids), copoly(ether-esters), polyalkylenes oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, poly(propylene fumarate), polyurethane, poly(ester urethane), poly(ether urethane), and blends and copolymers thereof. Suitable synthetic polymers for use in the present invention can also include biosynthetic polymers based on sequences found in collagen, laminin, glycosaminoglycans, elastin, thrombin, fibronectin, starches, poly(amino acid), gelatin, alginate, pectin, fibrin, oxidized cellulose, chitin, chitosan, tropoelastin, hyaluronic acid, silk, ribonucleic acids, deoxyribonucleic acids, polypeptides, proteins, polysaccharides, polynucleotides and combinations thereof.

For the purpose of this invention aliphatic polyesters include, but are not limited to, homopolymers and copolymers of lactide (which includes lactic acid, D- , L- and meso lactide); glycolide (including glycolic acid); ε-caprolactone; p-dioxanone (1,4-dioxan-2-one); trimethylene carbonate (1,3-dioxan-2-one); alkyl derivatives of trimethylene carbonate; δ-valerolactone; β-butyrolactone; γ-butyrolactone; ε-decalactone; hydroxybutyrate; hydroxyvalerate; 1,4-dioxepan-2-one (including its dimer 1,5,8,12-tetraoxacyclotetradecane-7,14-dione); 1,5-dioxepan-2-one; 6,6-dimethyl-1,4-dioxan-2-one; 2,5-diketomorpholine; pivalolactone; α, α diethylpropiolactone; ethylene carbonate; ethylene oxalate; 3-methyl-1,4-dioxane-2,5-dione; 3,3-diethyl-1,4-dioxan-2,5-dione; 6,6-dimethyl-dioxepan-2-one; 6,8-dioxabicycloctane-7-one and polymer blends thereof. Aliphatic polyesters used in the present invention can be homopolymers or copolymers (random, block, segmented, tapered blocks, graft, triblock, etc.) having a linear, branched or star structure. Other useful polymers include polyphosphazenes, co-, ter- and higher order mixed monomer based polymers made from L-lactide, D,L-lactide, lactic acid, glycolide, glycolic acid, para-dioxanone, trimethylene carbonate and ε-caprolactone.

In one embodiment, the device includes at least one natural polymer. Suitable examples of natural polymers include, but are not limited to, fibrin-based materials, collagen-based materials, hyaluronic acid-based materials, glycoprotein-based materials, cellulose-based materials, silks and combinations thereof.

In yet another embodiment, the device includes a naturally occurring extracellular matrix material (“ECM”), such as that found in the stomach, bladder, alimentary, respiratory, urinary, integumentary, genital tracts, or liver basement membrane of animals. Preferably, the ECM is derived from the alimentary tract of mammals, such as cows, sheep, dogs, cats, and most preferably from the intestinal tract of pigs. The ECM is preferably small intestine submucosa (“SIS”), which can include the tunica submucosa, along with basilar portions of the tunica mucosa, particularly the lamina muscularis mucosa and the stratum compactum.

In other embodiments of the present invention, the device can be formed from elastomeric copolymers such as, for example, polymers having an inherent viscosity in the range of about 1.2 dL/g to 4 dL/g, more preferably about 1.2 dL/g to 2 dL/g, and most preferably about 1.4 dL/g to 2 dL/g as determined at 25° C. in a 0.1 gram per deciliter (g/dL) solution of polymer in hexafluoroisopropanol (HFIP). Suitable elastomers also preferably exhibit a high percent elongation and a low modulus, while possessing good tensile strength and good recovery characteristics.

Exemplary biocompatible elastomers include, but are not limited to, elastomeric copolymers of ε-caprolactone and glycolide with a mole ratio of ε-caprolactone to glycolide of from about 35:65 to about 65:35, more preferably from 45:55 to 35:65; elastomeric copolymers of ε-caprolactone and lactide (including L-lactide, D-lactide, blends thereof, and lactic acid polymers and copolymers) where the mole ratio of ε-caprolactone to lactide is from about 95:5 to about 30:70 and more preferably from 45:55 to 30:70 or from about 95:5 to about 85:15; elastomeric copolymers of p-dioxanone (1,4-dioxan-2-one) and lactide (including L-lactide, D-lactide, blends thereof, and lactic acid polymers and copolymers) where the mole ratio of p-dioxanone to lactide is from about 40:60 to about 60:40; elastomeric copolymers of ε-caprolactone and p-dioxanone where the mole ratio of ε-caprolactone to p-dioxanone is from about from 30:70 to about 70:30; elastomeric copolymers of p-dioxanone and trimethylene carbonate where the mole ratio of p-dioxanone to trimethylene carbonate is from about 30:70 to about 70:30; elastomeric copolymers of trimethylene carbonate and glycolide (including polyglycolic acid) where the mole ratio of trimethylene carbonate to glycolide is from about 30:70 to about 70:30; elastomeric copolymers of trimethylene carbonate and lactide (including L-lactide, D-lactide, blends thereof, and lactic acid polymers and copolymers) where the mole ratio of trimethylene carbonate to lactide is from about 30:70 to about 70:30; and blends thereof. Other examples of suitable biocompatible elastomers are described in U.S. Pat. No. 5,468,253.

In another embodiment of the present invention, the device can be formed from an elastomer that is a copolymer of 35:65 ε-caprolactone and glycolide, formed in a dioxane solvent and including a polydioxanone mesh. In another embodiment, the elastomer used to form the tissue repair device can be a copolymer of 40:60 ε-caprolactone and lactide with a polydioxanone mesh. In yet another embodiment, the elastomer is a 50:50 blend of a 35:65 copolymer of ε-caprolactone and glycolide and 40:60 copolymer of ε-caprolactone and lactide. The polydioxanone mesh may be in the form of a one layer thick two-dimensional mesh or a multi-layer thick three-dimensional mesh.

In yet another embodiment of the present invention, the device can be formed from a polymeric foam component having pores with an open cell pore structure. The pore size can vary, but preferably, the pores are sized to allow tissue and vascular ingrowth. More preferably, the pore size is in the range of about 200 to 1000 microns, and even more preferably, in the range of about 50 to 500 microns. The polymeric foam component can, optionally, contain a reinforcing component, such as for example, the textiles disclosed above. In some embodiments where the polymeric foam component contains a reinforcing component, the foam component can be integrated with the reinforcing component such that the pores of the foam component penetrate the mesh of the reinforcing component and interlock with the reinforcing component.

It may also be desirable to use polymer blends to form a device which transitions from one composition to another composition in a gradient-like architecture. For example, by blending an elastomer of ε-caprolactone-co-glycolide with ε-caprolactone-co-lactide (e.g., with a mole ratio of about 5:95) a device may be formed that transitions from a softer spongy material to a stiffer more rigid material. Clearly, one skilled in the art will appreciate that other polymer blends may be used for similar gradient effects, or to provide different gradients (e.g., different absorption profiles, stress response profiles, different degrees of elasticity, or different porosities).

One of ordinary skill in the art will appreciate that the selection of a suitable material for forming the biocompatible tissue device of the present invention depends on several factors. These factors include in vivo mechanical performance; cell response to the material in terms of cell attachment, proliferation, migration and differentiation; biocompatibility; and optionally, bioabsorption (or bio-degradation) kinetics. Other relevant factors include the chemical composition, spatial distribution of the constituents, the molecular weight of the polymer, and the degree of crystallinity.

The differences in the absorption time under in vivo conditions can also be the basis for combining two different copolymers when forming the device of the present invention. For example, a copolymer of 35:65 ε-caprolactone and glycolide (a relatively fast absorbing polymer) can be blended with 40:60 ε-caprolactone and L-lactide copolymer (a relatively slow absorbing polymer) to form a biocompatible scaffold. Depending upon the processing technique used, the two constituents can be either randomly inter-connected bicontinuous phases, or the constituents could have a gradient-like architecture in the form of a laminate-type composite with a well integrated interface between the two constituent layers.

The meniscal repair device used to form the implant can also include a reinforcing material comprised of any absorbable or non-absorbable textile having, for example, woven, knitted, warped knitted (i.e., lace-like), non-woven, and braided structures. In one embodiment, the reinforcing material has a mesh-like structure. In any of the above structures, mechanical properties of the material can be altered by changing the density or texture of the material, the type of knit or weave of the material, the thickness of the material, or by embedding particles in the material. The mechanical properties of the material may also be altered by creating sites within the mesh where the fibers are physically bonded with each other or physically bonded with another agent, such as, for example, an adhesive or a polymer.

The fibers used to make the reinforcing component can include monofilaments, yarns, threads, braids, or bundles of fibers. These fibers can be made of any biocompatible material including bioabsorbable materials such as polylactic acid (PLA), polyglycolic acid (PGA), polycaprolactone (PCL), polydioxanone (PDO), trimethylene carbonate (TMC), copolymers or blends thereof. These fibers can also be made from any biocompatible materials based on natural polymers including silk and collagen-based materials. These fibers can also be made of any biocompatible fiber that is nonresorbable, such as, for example, polyethylene, polyethylene terephthalate, poly(tetrafluoroethylene), polycarbonate, polypropylene and poly(vinyl alcohol).

The device, as well as any reinforcing material, may also be formed from a thin, perforation-containing elastomeric sheet with pores or perforations to allow tissue ingrowth. Such a sheet could be made of blends or copolymers of polylactic acid (PLA), polyglycolic acid (PGA), polycaprolactone (PCL), and polydioxanone (PDO).

A person skilled in the art will appreciate that one or more layers of the reinforcing material may be used to reinforce the composite implant of the invention. In addition, biodegradable textiles, such as, for example, meshes, of the same structure and chemistry or different structures and chemistries can be overlaid on top of one another to fabricate biocompatible scaffolds with superior mechanical strength.

In one embodiment, the device of the present invention includes a high-density, nonwoven polymeric material. Preferably, the nonwoven material includes flexible, porous structures produced by interlocking layers or networks of fibers, filaments, or film-like filamentary structures. The polymeric material used to construct the nonwoven can include the bioabsorbable synthetic polymer materials listed above. The nonwoven may additionally include a biocompatible foam for reinforcing the device.

The devices of the present invention can preferably include a source of viable tissue. The source of viable tissue can vary, and the tissue source can have a variety of configurations. In one embodiment, however, the tissue is in the form of finely minced tissue fragments, which enhance the effectiveness of the regrowth and healing response. In another embodiment, the viable tissue can be in the form of a tissue slice or strip that harvested from healthy tissue that contains viable cells capable of tissue regeneration and/or remodeling. The tissue slice is preferably harvested to have a geometry that is suitable for implantation at the site of the injury or defect, and the harvested tissue slice is preferably dimensioned to allow the viable cells contained within the tissue slice to migrate out and proliferate and integrate with tissue surrounding the repair site.

The term “slice,” as used herein, refers to a thin section, strip or sliver derived from any of the tissue types described above and used to construct the tissue implant. Preferably, the tissue slice has a thickness less than about 1 mm, and more preferably has a thickness in the range of about 200 μm to about 500 μm. A thin profile ensures proper migration of the cells out of the tissue slice. It is understood, however, that the tissue slice can have any length or width appropriate for implantation at the defect, since these parameters do not greatly affect cell migration out of the tissue slice.

Where a tissue fragment is used with the device of the present invention, the particle size of each tissue fragment can also vary. By way of non-limiting example, the tissue size can be in the range of about 0.1 and 3 mm³, in the range of about 0.5 and 1 mm³, in the range of about 1 to 2 mm³, or in the range of about 2 to 3 mm³, but preferably the tissue particle is less than 1 mm³.

Suitable tissue from which the tissue source can be derived includes, for example, cartilage tissue, meniscal tissue, ligament tissue, tendon tissue, skin tissue, muscle tissue, periosteal tissue, pericardial tissue, synovial tissue, nerve tissue, fat tissue, kidney tissue, bone marrow, liver tissue, bladder tissue, pancreas tissue, spleen tissue, intervertebral disc tissue, embryonic tissue, periodontal tissue, vascular tissue, blood, and combinations thereof. The tissue used to construct the tissue implant can be autogeneic tissue, allogeneic tissue, or xenogeneic tissue. In a preferred embodiment, the viable tissue is autogeneic meniscal tissue.

The viable tissue, bioactive agents can also optionally be combined with a variety of other materials, including carriers, such as a gel-like carrier or an adhesive. Alternately, the gel-like carrier or adhesive may be used alone as a mean of maintaining channel patency.

By way of non-limiting example, the gel-like carrier can be a biological or synthetic hydrogel such as hyaluronic acid, fibrin glue, fibrin clot, collagen gel, collagen-based adhesive, alginate gel, crosslinked alginate, chitosan, synthetic acrylate-based gels, platelet rich plasma (PRP), platelet poor plasma (PPP), PRP clot, PPP clot, blood, blood clot, blood component, blood component clot, Matrigel, agarose, chitin, chitosan, polysaccharides, poly(oxyalkylene), a copolymer of poly(ethylene oxide)-poly(propylene oxide), poly(vinyl alcohol), laminin, elastin, proteoglycans, solubilized basement membrane, or combinations thereof. Suitable adhesives include, but are not limited to, hyaluronic acid, fibrin glue, fibrin clot, collagen gel, collagen-based adhesive, alginate gel, crosslinked alginate, gelatin-resorcin-formalin-based adhesive, mussel-based adhesive, dihydroxyphenylalanine (DOPA)-based adhesive, chitosan, transglutaminase, poly(amino acid)-based adhesive, cellulose-based adhesive, polysaccharide-based adhesive, synthetic acrylate-based adhesives (such as cyanoacrylates), platelet rich plasma (PRP), platelet poor plasma (PPP), PRP clot, PPP clot, blood, blood clot, blood component, blood component clot, polyethylene glycol-based adhesive, Matrigel, Monostearoyl Glycerol co-Succinate (MGSA), Monostearoyl Glycerol co-Succinate/polyethylene glycol (MGSA/PEG) copolymers, laminin, elastin, proteoglycans, and combinations thereof.

The viable tissue can also be contacted with a matrix-digesting enzyme to facilitate tissue migration out of the extracellular matrix surrounding the viable tissue. The enzymes can be used to increase the rate of cell migration out of the extracellular matrix and into the tissue defect or injury, or scaffold material. Suitable matrix-digesting enzymes that can be used in the present invention include, but are not limited to, collagenase, chondroitinase, trypsin, elastase, hyaluronidase, peptidase, thermolysin, matrix metalloproteinase, gelatinase and protease. Preferably, the concentration of minced tissue particles in the gel-carrier is in the range of approximately 1 to 1000 mg/cm³, and more preferably in the range of about 1 to 200 mg/cm³.

In another embodiment of the present invention, a bioactive agent may be incorporated within and/or applied to the meniscal repair device, and/or it can be applied to the viable tissue. Preferably, the bioactive agent is incorporated within, or coated on, the device prior to the addition of viable tissue. The bioactive agent(s) can be selected from among a variety of effectors that, when present at the site of injury, promote healing and/or regeneration of the affected tissue. In addition to being compounds or agents that actually promote or expedite healing, the effectors may also include compounds or agents that prevent infection (e.g., antimicrobial agents and antibiotics), compounds or agents that reduce inflammation (e.g., anti-inflammatory agents), compounds that prevent or minimize adhesion formation, such as oxidized regenerated cellulose (e.g., INTERCEED® and SURGICEL®, available from Ethicon, Inc.), hyaluronic acid, and compounds or agents that suppress the immune system (e.g., immunosuppressants).

By way of non-limiting example, other types of effectors present within the implant of the present invention can include heterologous or autologous growth factors, proteins (including matrix proteins), peptides, antibodies, enzymes, platelets, platelet rich plasma, glycoproteins, hormones, cytokines, glycosaminoglycans, nucleic acids, analgesics, viruses, virus particles, and cell types. It is understood that one or more effectors of the same or different functionality may be incorporated within the implant.

Examples of suitable effectors include the multitude of heterologous or autologous growth factors known to promote healing and/or regeneration of injured or damaged tissue. These growth factors can be incorporated directly into the meniscal repair device, or alternatively, the device can include a source of growth factors, such as for example, platelets. “Bioactive agents,” as used herein, can include one or more of the following: chemotactic agents; therapeutic agents (e.g., antibiotics, steroidal and non-steroidal analgesics and anti-inflammatories, anti-rejection agents such as immunosuppressants and anti-cancer drugs); various proteins (e.g., short term peptides, bone morphogenic proteins, glycoprotein and lipoprotein); cell attachment mediators; biologically active ligands; integrin binding sequence; ligands; various growth and/or differentiation agents and fragments thereof (e.g., epidermal growth factor (EGF), hepatocyte growth factor (HGF), vascular endothelial growth factors (VEGF), fibroblast growth factors (e.g., bFGF), platelet derived growth factors (PDGF), insulin derived growth factor (e.g., IGF-1, IGF-II) and transforming growth factors (e.g., TGF-βI-III), parathyroid hormone, parathyroid hormone related peptide, bone morphogenic proteins (e.g., BMP-2, BMP-4; BMP-6; BMP-7; BMP-12; BMP-13; BMP-14), sonic hedgehog, growth differentiation factors (e.g., GDF5, GDF6, GDF8), recombinant human growth factors (e.g., MP52, (aka rhGDF-5)), cartilage-derived morphogenic proteins (CDMP-1; CDMP-2; CDMP-3); small molecules that affect the upregulation of specific growth factors; tenascin-C; hyaluronic acid; chondroitin sulfate; fibronectin; decorin; thromboelastin; thrombin-derived peptides; heparin-binding domains; heparin; heparan sulfate; DNA fragments and DNA plasmids. Suitable effectors likewise include the agonists and antagonists of the agents described above. The growth factor can also include combinations of the growth factors described above. In addition, the growth factor can be autologous growth factor that is supplied by platelets in the blood. In this case, the growth factor from platelets will be an undefined cocktail of various growth factors. If other such substances have therapeutic value in the orthopaedic field, it is anticipated that at least some of these substances will have use in the present invention, and such substances should be included in the meaning of “bioactive agent” and “bioactive agents” unless expressly limited otherwise.

Biologically derived agents, suitable for use as effectors, include one or more of the following: cartilage (autograft, allograft and xenograft), including, for example, meniscal tissue, and derivatives; ligament (autograft, allograft and xenograft) and derivatives; derivatives of intestinal tissue (autograft, allograft and xenograft), including for example submucosa; derivatives of stomach tissue (autograft, allograft and xenograft), including for example submucosa; derivatives of bladder tissue (autograft, allograft and xenograft), including for example submucosa; derivatives of alimentary tissue (autograft, allograft and xenograft), including for example submucosa; derivatives of respiratory tissue (autograft, allograft and xenograft), including for example submucosa; derivatives of genital tissue (autograft, allograft and xenograft), including for example submucosa; derivatives of liver tissue (autograft, allograft and xenograft), including for example liver basement membrane; derivatives of skin tissue; platelet rich plasma (PRP), platelet poor plasma, bone marrow aspirate, insulin derived growth factor, whole blood, fibrin and blood clot. Purified ECM and other collagen sources are also appropriate biologically derived agents. If other such substances have therapeutic value in the orthopaedic field, it is anticipated that at least some of these substances will have use in the present invention, and such substances should be included in the meaning of “biologically derived agent” and “biologically derived agents” unless expressly limited otherwise.

Biologically derived agents also include bioremodelable collageneous tissue matrices. The terms “bioremodelable collageneous tissue matrix” and “naturally occurring bioremodelable collageneous tissue matrix” include matrices derived from native tissue selected from the group consisting of skin, artery, vein, pericardium, heart valve, dura mater, ligament, cartilage, bladder, liver, stomach, fascia and intestine, whatever the source. Although the term “naturally occurring bioremodelable collageneous tissue matrix” is intended to refer to matrix material that has been cleaned, processed, sterilized, and optionally crosslinked, it is not within the definition of a naturally occurring bioremodelable collageneous tissue matrix to purify the natural fibers and reform a matrix material from purified natural fibers.

The proteins that may be present within the implant include proteins that are manufactured by a cell (secreted or cytoplasmic) or other biological source, such as for example, a platelet, which is housed within the implant, as well as those that are present within the implant in an isolated form. The isolated form of a protein typically is one that is about 55% or greater in purity, i.e., isolated from other cellular proteins, molecules, debris, etc. More preferably, the isolated protein is one that is at least 65% pure, and most preferably one that is at least about 75 to 95% pure. Notwithstanding the above, one of ordinary skill in the art will appreciate that proteins having a purity below about 55% are still considered to be within the scope of this invention. As used herein, the term “protein” embraces glycoproteins, lipoproteins, proteoglycans, peptides, and fragments thereof. Examples of proteins useful as effectors include, but are not limited to, pleiotrophin, endothelin, tenascin, fibronectin, fibrinogen, vitronectin, V-CAM, I-CAM, N-CAM, selectin, cadherin, integrin, laminin, actin, myosin, collagen, microfilament, intermediate filament, antibody, elastin, fibrillin, and fragments thereof.

Glycosaminoglycans, highly charged polysaccharides which play a role in cellular adhesion, may also serve as effectors according to the present invention. Exemplary glycosaminoglycans useful as effectors include, but are not limited to, heparan sulfate, heparin, chondroitin sulfate, dermatan sulfate, keratan sulfate, hyaluronan (also known as hyaluronic acid), and combinations thereof.

The meniscal repair device of the present invention can also have cells incorporated therein to serve as effectors. The cells may be cultured or uncultured. Suitable cell types that can serve as effectors according to this invention include, but are not limited to, osteocytes, osteoblasts, osteoclasts, fibroblasts, stem cells, pluripotent cells, chondrocyte progenitors, chondrocytes, fibrochondrocytes, endothelial cells, macrophages, leukocytes, adipocytes, monocytes, plasma cells, mast cells, umbilical cord cells, stromal cells, mesenchymal stem cells, epithelial cells, myoblasts, tenocytes, ligament fibroblasts, neurons, bone marrow cells, synoviocytes, embryonic stem cells; precursor cells derived from adipose tissue; peripheral blood progenitor cells; stem cells isolated from adult tissue; genetically transformed cells; a combination of chondrocytes and other cells; a combination of osteocytes and other cells; a combination of synoviocytes and other cells; a combination of bone marrow cells and other cells; a combination of mesenchymal cells and other cells; a combination of stromal cells and other cells; a combination of stem cells and other cells; a combination of embryonic stem cells and other cells; a combination of precursor cells isolated from adult tissue and other cells; a combination of peripheral blood progenitor cells and other cells; a combination of stem cells isolated from adult tissue and other cells; and a combination of genetically transformed cells and other cells. If other cells are found to have therapeutic value in the orthopaedic field, it is anticipated that at least some of these cells will have use in the present invention, and such cells should be included within the meaning of “cell” and “cells” unless expressly limited.

Cells typically have at their surface receptor molecules which are responsive to a cognate ligand (e.g., a stimulator). A stimulator is a ligand which when in contact with its cognate receptor induce the cell possessing the receptor to produce a specific biological action. For example, in response to a stimulator (or s ligand) a cell may produce significant levels of secondary messengers, like Ca⁺², which then will have subsequent effects upon cellular processes such as the phosphorylation of proteins, such as (keeping with our example) protein kinase C. In some instances, once a cell is stimulated with the proper stimulator, the cell secretes a cellular messenger usually in the form of a protein (including glycoproteins, proteoglycans, and lipoproteins). This cellular messenger can be an antibody (e.g., secreted from plasma cells), a hormone, (e.g., a paracrine, autocrine, or exocrine hormone), a cytokine, or natural or synthetic fragments thereof.

The meniscal repair device of the invention can also be used with gene therapy techniques in which nucleic acids, viruses, or virus particles deliver a gene of interest, which encodes at least one gene product of interest, to specific cells or cell types. Accordingly, the biological effector can be a nucleic acid (e.g., DNA, RNA, or an oligonucleotide), a virus, a virus particle, or a non-viral vector. The viruses and virus particles may be, or may be derived from, DNA or RNA viruses. The gene product of interest is preferably selected from the group consisting of proteins, polypeptides, interference ribonucleic acids (iRNA) and combinations thereof.

Once the applicable nucleic acids and/or viral agents (i.e., viruses or viral particles) are incorporated into the meniscal repair device, the device can then be implanted to elicit a type of biological response. The nucleic acid or viral agent can then be taken up by the cells and any proteins that they encode can be produced locally by the cells. In one embodiment, the nucleic acid or viral agent can be taken up by the cells within the tissue fragment of the minced tissue suspension, or, in an alternative embodiment, the nucleic acid or viral agent can be taken up by the cells in the tissue surrounding the site of the injured tissue. One skilled in the art will recognize that the protein produced can be a protein of the type noted above, or a similar protein that facilitates an enhanced capacity of the tissue to heal an injury or a disease, combat an infection, or reduce an inflammatory response. Nucleic acids can also be used to block the expression of unwanted gene product that may impact negatively on a tissue repair process or other normal biological processes. DNA, RNA and viral agents are often used to accomplish such an expression blocking function, which is also known as gene expression knock out.

One skilled in the art will appreciate that the identity of the bioactive agent may be determined by a surgeon, based on principles of medical science and the applicable treatment objectives. It is understood that the bioactive agent or effector of the tissue repair implant can be incorporated within the tissue repair device or on the surface of the tissue repair device before, during, or after manufacture of the device, or before, during, or after the surgical placement of the device.

By way of example, a bioactive agent can be incorporated into the meniscal repair device by placing the meniscal repair device in a suitable container comprising the bioactive agent. After an appropriate amount time and under suitable conditions, the device will become impregnated with the bioactive agent. Alternatively, the bioactive agent can be incorporated within the meniscal repair device by, for example, using an appropriately gauged syringe to inject the biological agent(s) into the device. Other methods well known to those skilled in the art can be applied in order to load the device with an appropriate bioactive agent. Such techniques include mixing, coating, pressing, spreading, centrifuging and placing the bioactive agent into the device. Alternatively, the bioactive agent can be mixed with a gel-like carrier prior to injection into the device.

In another embodiment, a surgically implanted meniscal repair device devoid of any bioactive agent can be infused with biological agent(s), or an implant including at least one bioactive agent can be augmented with a supplemental quantity of the bioactive agent. One method of incorporating a bioactive agent within a surgically implanted device is by injection using an appropriately gauged syringe.

The amount of the bioactive agent included with a meniscal repair device will vary depending on a variety of factors, including the size of the device, the porosity, the identity of the bioactive component, and the intended purpose of the tissue repair implant. One skilled in the art can readily determine the appropriate quantity of bioactive agent to include for a given application in order to facilitate and/or expedite the healing of tissue.

After positioning the meniscal repair device of the present invention within a patient, the device may be fastened within the damaged or torn meniscal tissue. In one embodiment, the repair device is fixed to adjacent tissue such that the repair device is anchored in place. The repair device can be anchored to soft and/or hard tissue such as meniscal tissue or tibial tissue. In another embodiment, the repair device may additionally or alternatively be fixed to meniscal tissue to hold damaged or loose tissue in position. Joining the repair device with loose meniscal tissue provides support to the damaged tissue area and thereby facilitates rapid healing. A person skilled in the art will appreciate that a variety of techniques can be used to fix the device to hard and/or soft tissue, such as, for example, an interference fit, suture, glue, staple, tissue tack, arrow, pins, and/or other known surgical fixation techniques.

The present invention also includes methods of surgically repairing meniscal defects with the meniscal repair device of the present invention by positioning at least a portion of the repair device in contact with a defect in a meniscus. In one embodiment, one end of the device is positioned in or across a meniscal tissue tear, and a second end is positioned in contact with a tibial surface. A person skilled in the art will appreciate that contact with the tibial surface includes placing the device in proximity to the tibial surface when biological materials, such as blood, form a thin layer between the repair device and the tibial surface. The device is then fixed in position, preferably with sutures extending through the device and meniscus.

The meniscal repair device can be used to fix or enhance healing of all types of meniscal tissue defects. For example, the device can be used to repair tears such as bucket handle (vertical), longitudinal, horizontal, degenerative, radial, flap, and parrot beak tears. In another example, the meniscal repair device may enhance regeneration of meniscal tissue following partial meniscectomy.

In one embodiment, the method can additionally include the step of inducing bleeding at or near the tissue defect to promote the flow of biological materials necessary for regenerating meniscal tissue. Preferably, the meniscus, and/or synovium is rasped or any other technique that the surgeon may use to create bleeding in the joint prior to or following implantation the tissue repair device. In another embodiment, a portion of the synovium may be pulling into a portion of the device of this invention to provide a source of blood, cells or nutrients.

Additionally, the devices and methods of this invention may be used in conjunction with other scaffolds or other tissue repair devices known in the art and comprised of materials know in the art. Thus, a scaffold or other tissue repair device may be placed at the site of the defect (i.e., within, across, over or under) and the device of this invention may contact or intersect the scaffold or other tissue repair device at any angle.

FIG. 5 depicts another embodiment of this invention wherein solid or non-solid material 8 is used in the meniscal repair channel to repair tear 20 of meniscus 1.

Thus, FIG. 5 supports a method of surgically repairing meniscal defects, comprising:

creating a channel or slice in the meniscus between the defect in the meniscus and a vascularized portion of the meniscus, and

providing a material in the channel or slice to permit controlled patency of the channel or slice to enhance travel of cells and nutrients from the vascularized portion of the meniscus to the defect in the meniscus and thereby encourage healing of the meniscal defect.

The materials may be an anticoagulant, enzyme, or gel, either alone or in combination with a carrier. The material may further be a solid material such as the solid, lumen stents, or porous matrix scaffolds described above. Furthermore, bioactive agents as described above may be incorporated into these materials and the forgoing descriptions and embodiments of any of the foregoing elements or features are herein incorporated by reference.

It should be understood that the foregoing disclosure and description of the present invention are illustrative and explanatory thereof and various changes in the size, shape and materials as well as in the description of the preferred embodiment may be made without departing from the spirit of the invention.

What is claimed is:

1. A biocompatible meniscal repair device, comprising:
a biocompatible tissue repair stent adapted to be placed in contact with a defect in a meniscus and adapted to contact a vascularized portion of the meniscus and communicate biological materials from the vascularized portion of the meniscus to the tissue defect in the meniscus.

2. The repair device of claim 1, wherein the stent provides a conduit that enables blood, cells or nutrients to migrate from the vascularized portion of the meniscus to the tissue defect in the meniscus.

3. The repair device of claim 2, wherein the stent is adapted to contact the synovium.

4. The repair device of claim 1, wherein the tissue repair stent is constructed from a bioabsorbable material.

5. The repair device of claim 1, wherein the tissue repair stent is constructed from a non-bioabsorbable material.

6. The repair device of claim 1, wherein the tissue repair stent is in-situ expandable.

7. The repair device of claim 1, wherein the tissue repair stent is a non-porous tube.

8. The repair device of claim 1, wherein the tissue repair stent is a tube with porous walls.

9. The repair device of claim 1, wherein the tissue repair stent comprises a mesh.

10. The repair device of claim 4 wherein the tissue repair stent includes at least one polymer derived from monomers selected from the group consisting of caprolactone, glycolide, lactide, dioxanone and mixtures thereof.

11. The repair device of claim 1, wherein the tissue repair stent comprises polydioxanone.

12. The repair device of claim 1, wherein the tissue repair stent comprises a copolymer of glycolide and L-lactide.

13. The repair device of claim 1, wherein the tissue repair stent is formed from at least one material selected from the group consisting of natural polymers, synthetic polymers, and combinations thereof.

14. The device of claim 13 wherein the material is SIS.

15. The repair device of claim 1, further comprising a viable tissue disposed on or within the tissue repair stent and effective to integrate with native tissue adjacent to the tissue repair stent.

16. The repair device of claim 1, further comprising at least one bioactive substance effective to stimulate cell growth.

17. The repair device of claim 16, wherein the bioactive substance is selected from the group consisting of bone morphogenic proteins, VEGF, PDGF, TGF-β, platelet rich plasma, cartilage-derived morphogenic proteins, recombinant human growth factors, and combinations thereof.

18. The repair device of claim 17, wherein the bioactive substance is selected from the group consisting of rhGDF-5, BMP-2, BMP-7, and CDMP-1.

19. The repair device of claim 1, wherein the tissue repair stent is a porous matrix capable encouraging migration of blood, cells, or nutrients from one end of the stent to another end.

20. The repair device of claim 19, wherein the matrix comprises a bioactive agent.

21. The repair device of claim 19, wherein the stent has a hollow core.

22. The repair device of claim 1, wherein the stent has barbs on its outer surface to encourage fixation within the meniscus.

23. The repair device of claim 1, wherein the stent is is made of a shape memory material.

24. The repair device of claim 23, wherein the shape memory material comprises a metal.

25. The repair device of claim 24, wherein the shape memory material is nitinol.

26. The repair device of claim 23, wherein the shape memory material comprises a polymer.

27. A method of surgically repairing meniscal defects, comprising:
providing a tissue repair stent;
positioning a first end of the tissue repair stent in contact with the defect in the meniscus while positioning a second end of the stent in contact with a vascularized portion of the meniscus, wherein the stent allows blood, cells or nutrients to migrate from the vascularized portion of the meniscus to the defect in the meniscus and thereby encourage healing of the meniscus.

28. The method of claim 27, further comprising the step of rasping the meniscus before or after positioning the stent.

29. The method of claim 27, further comprising placing the second end of the stent in contact with the synovium.

30. The method of claim 29, further comprising the step of rasping the synovium before or after positioning the stent.

31. The method of claim 29, wherein the stent provides a conduit that enables blood, cells or nutrients to migrate from the synovium to the tissue defect in a meniscus.

32. The method of claim 27, wherein the blood, cells, or nutrients are provided from an exogenous source.

33. A method of surgically repairing meniscal defects, comprising:
creating a channel or slice in the meniscus between the defect in the meniscus and a vascularized portion of the meniscus, and
providing a material in the channel or slice to permit controlled patency of the channel or slice to enhance migration of blood, cells, or nutrients from the vascularized portion of the meniscus to the defect in is the meniscus and thereby encourage healing of the meniscal defect.

34. The method of claim 33 wherein the material is an anticoagulant, enzyme, or gel.

35. The method of claim 34, wherein the material is a gel.

36. The method of claim 34, wherein the anticoagulant is selected from the group consisting of acid citrate dextrose (ACD), citrate phosphate dextrose (CPD), citrate, heparin, potassium oxalate, potassium citrate, potassium ethylene diamine tetraacetic acid (EDTA) and combinations thereof.

37. The method of claim 34, wherein the enzyme is selected from the group consisting of collagenase, chondroitinase, trypsin, elastase, hyaluronidase, peptidase, thermolysin, matrix metalloproteinase, gelatinase, protease, and combinations thereof.

38. The method of claim 33, wherein the material further comprises a bioactive substance selected from the group consisting of bone morphogenic proteins, VEGF, PDGF, TGF-β, platelet rich plasma, cartilage-derived morphogenic proteins, recombinant human growth factors, and combinations thereof.

39. The method of claim 38, wherein the bioactive substance is selected from the group consisting of rhGDF-5, BMP-2, BMP-7, and CDMP-1.

40. The method of claim 38, wherein the bioactive agent is in a gel carrier.
